SEQUENCE LISTING (28 sequences)

SEQ ID NO: 1 - DNA, 43 bp, artificial sequence
  primer
gacagagctcttcatcatggattctatattatcaaaacagctg

SEQ ID NO: 2 - DNA, 18 bp, artificial sequence
  primer
tgaggcatccattgctgc

SEQ ID NO: 3 - DNA, 19 bp, artificial sequence
  primer
gctgggcctttgatctctc

SEQ ID NO: 4 - DNA, 18 bp, artificial sequence
  primer
ctcccgatgaccatccag

SEQ ID NO: 5 - DNA, 22 bp, artificial sequence
  primer
gaggaaagaattgttcaatcgg

SEQ ID NO: 6 - DNA, 22 bp, artificial sequence
  primer
aatggtcacggataaccatagc

SEQ ID NO: 7 - DNA, 22 bp, artificial sequence
  primer
aagcaactctagctcacacccc

SEQ ID NO: 8 - DNA, 36 bp, artificial sequence
  primer
gacagagctcttctggtcttagcctagcatgtcatc

SEQ ID NO: 9 - DNA, 46 bp, artificial sequence
  primer
gacagagctcttctaaatgtatgttttattaacaattctaatctcg

SEQ ID NO: 10 - DNA, 18 bp, artificial sequence
  primer
tcaaacaagcctctgccc

SEQ ID NO: 11 - DNA, 21 bp, artificial sequence
  primer
gtgaacagggaaataggatgg

SEQ ID NO: 12 - DNA, 37 bp, artificial sequence
  primer
gacagagctcttcctgatctatgaggccttcttagtg

SEQ ID NO: 13 - DNA, 39 bp, Artificial Sequence
  primer
gacagagctcttcataatggacaactatcaagagcttgc

SEQ ID NO: 14 - DNA, 36 bp, artificial sequence
  primer
gacagagctcttccaagcagcaaaagaggacatttc

SEQ ID NO: 15 - DNA, 80 bp, artificial sequence
  primer
gacagacgtctcctatagacacaaagacggtgcattaaatgaagagcggtaccgctcttcatcagtacgtgtaaaagcaa

SEQ ID NO: 16 - DNA, 80 bp, artificial sequence
  primer
tgacctatctgtacaatacttacataattaggtttgcttatgtctacttatttcaacatattgcttttacacgtactgat

SEQ ID NO: 17 - DNA, 80 bp, artificial sequence
  primer
agtattgtacagataggtcaaattattggaatatccaagcttagaaacttatgcaataatactttagatgtaagcttagt

SEQ ID NO: 18 - DNA, 80 bp, artificial sequence
  primer
actagaatattcacaagcacttgagactgctgctgcctcaccccaccaccccaaattacaactaagcttacatctaaagt

SEQ ID NO: 19 - DNA, 80 bp, artificial sequence
  primer
gtgcttgtgaatattctagttggcgtaatcgtcttttgccagattagctgggaattaaactaactctttgaagttgcacc

SEQ ID NO: 20 - DNA, 48 bp, artificial sequence
  primer
tctgtccgtctccacccacacaaagaccggtgcaacttcaaagagtta

SEQ ID NO: 21 - DNA, 39 bp, artificial sequence
  primer
gacagacgtctcctattacacaaagaccggtgcaacttc

SEQ ID NO: 22 - DNA, 36 bp, artificial sequence
  primer
gacagacgtctccgggacacaaagacggtgcattaa

SEQ ID NO: 23 - DNA, 41 bp, artificial sequence
  primer
gacagagctcttcaaaatgacttcgaaagtttatgatccag

SEQ ID NO: 24 - DNA, 43 bp, artificial sequence
  primer
gacagagctcttcttgacttattgttcatttttgagaactcgc

SEQ ID NO: 25 - DNA, 10608 bp, Artificial Sequence
  expression plasmid
gtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacttttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcaattccctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctggggatctagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatctttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagctcctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgatcggatcccgggtaccgagctcggatccagtacccttcaccgacagagctcttcatcatggattctatattatcaaaacagctggttgacaagactggttttgttagagtgccaatcaagcattttgactgtacaatgctaactctggcacttccaacatttgatgtttccaagatggtagatagaattaccatagacttcaatctggatgatatacaaggagcatctgaaataggctcaactttgctaccctccatgtcgatagatgtggaagatatggccaattttgttcacgatttcacctttggccacttagctgacaagactgacagactgttaatgcgtgagtttcccatgatgaatgacgggtttgatcatttgagccctgacatgatcattaaaactacatctggcatgtacaacatcgttgagttcaccacctttaggggagatgaaagaggtgcattccaggctgccatgactaaactcgctaagtatgaggttccttgtgagaacagatctcagggcaggactgttgttctttatgttgttagtgcttatcggcatggtgtatggtctaatctggagctagaggactctgaagcagaggagatggtttataggtacagacttgctcttagtgtgatggatgagctaaggaccttgttcccagaactgtcatccacagatgaggaactagggaagactgagagagagttgctagccatggtctcctccatccaaataaattggtcagtcacagaatctgtgtttccacccttcagcagagaaatgtttgacaggtttagatcctcccctcccgattcagagtatatcacgaggatagtgagcagatgcctaataaattctcaagagaaactcatcaatagttccttctttgctgaagggaatgataaggctctgagattttcaaaaaacgctgaagagtgttccttggcagtagagagagccttaaatcagtatagagcagaagacaaccttagggacctcaatgaccacaagtcaactattcagctgcctccctggctgtcctatcatgatgtcgatggcaaagatctgtgccctcttcagggactagatgtgagaggggaccatcccatgtgcaacttgtggagggaagtggtcacctctgcaaacctagaggagattgagaggatgcacgatgatgcagcagcagaacttgagtttgccctttcgggagtaaaggacaggccagatgagagaaacagataccatagagtccacctaaatatgggctcagatgatagtgtctacatagctgctttaggagttaatggaaagaagcataaagcagacactttagtgcaacaaatgagagacaggagtaaacagcctttctccccagaccacgatgtggatcacatatctgaatttctctctgcatgctctagtgacttgtgggcaacagatgaggacctgtacagccctctctcttgtgataaagagcttagattggcagcccagaggattcatcagccatccttgtcagaaaggggtttcaatgagatcataacagagcactacaaattcatgggaagtaggataggttcatggtgccaaatggtcagcttgataggagctgagctatcagcttctgttaaacaacatgtcaagcctaactactttgtgattaaacgactactaggttctgggattttcttgctaatcaagcccacttccagcaaaagccatatatttgtgtcttttgcaattaagcgctcttgctgggcctttgatctctccacttccagggttttcaagccctacatagatgctggggatctgttagttactgactttgtttcttataagctaagcaagcttaccaacctctgcaagtgcgtttcattaatggagtcctccttctcattctgggcagaagcatttggaattccaagctggaactttgttggtgacttgttcaggtcttcagactctgcagcaatggatgcctcatacatgggcaaactttctttattaacccttttggaagacaaagcagcaactgaagagttacagactattgcaagatatataatcatggagggctttgtctcgcccccagaaatcccaaaacctcacaagatgacctctaagtttcctaaggttctcaggtcagagctgcaggtttacttattaaactgcttatgcagaactatccagagaatagcaggtgagcccttcattcttaagaagaaggatgggtctatatcctggggtggcatgttcaatcctttttcagggcgtccactgcttgatatgcaaccactcatcagctgttgttacaatggttactttaaaaataaagaagaagagactgagccttcgtccctttctgggatgtataagaaaatcatagaacttgagcaccttagaccacagtcagatgccttcttgggttacaaagatccagaacttcccagaatgcatgagttcagtgtttcctacttgaaggaggcttgcaatcatgctaagctagtcttgaggagcctctatggacagaatttcatggagcagatagacaaccagattattcgagagctcagtgggttgactctagaaaggttggccacacttaaggccacaagcaactttaatgagaattggtatgtctataaggatgtagcagacaaaaactacacaagggataaattattagtgaagatgtcaaaatatgcctctgagggaaagagcctagctatccagaagtttgaggattgtatgaggcagatagagtcacaaggatgcatgcatatttgtttgtttaagaaacaacagcatggaggtctgagagagatctatgtgatgggtgcagaggaaagaattgttcaatcggtggtggagacaatagccaggtccatagggaagttctttgcttctgataccctctgtaacccccccaataaagtgaaaattcctgagacacatggcatcagggcccggaagcaatgtaaggggcctgtgtggacttgtgcaacatcagatgatgcaaggaagtggaaccaaggccattttgttacaaagtttgccctcatgctgtgtgagttcacctctcctaaatggtggccgctgatcattaggggatgctcaatgtttaccaggaaaaggatgatgatgaatttgaattatcttaagatcctggatggtcatcgggagcttgatattagagatgactttgtaatggatctcttcaaagcttatcatggcgaggcagaagttccatgggcctttaaaggcaaaacatatttggaaaccacaacagggatgatgcagggaatactgcattatacttcctcactattacacaccattcaccaagaatacatccggtccttgtcctttaagatattcaacctgaaggttgctcctgagatgagcaagagcctggtttgtgacatgatgcaaggatcagatgatagtagtatgctaatcagcttcccagctgatgatgagaaggttcttaccagatgcaaagtggccgcagctatatgcttccgcatgaagaaggagctgggagtgtaccttgccatttacccctcagagaagtccacagcaaacacagattttgtgatggagtacaattctgaattttatttccacacccagcatgttagaccaacgatcaggtggattgcagcttgttgcagcctgccagaagtggaaacactagtagcccgccaggaagaggcctctaacctaatgacttcagttactgagggaggtgggtcattctccttagctgcaatgattcagcaagctcagtgtactctccattacatgctgatgggcatgggagtgtctgagctattcttagagtataagaaggcagtgctgaagtggaatgaccctggcctgggtttcttcctgcttgacaatccttatgcgtgcggattgggaggtttcagatttaatctcttcaaagctatcaccagaactgatttgcagaagctatatgctttcttcatgaagaaggtcaagggctcagctgctagggactgggcagatgaagatgtcaccatcccagaaacgtgtagcgtgagcccaggtggcgctctaattcttagctcctctctaaagtggggatctaggaagaagtttcagaaattgagagaccgtttgaacataccagagaactggattgaactaataaatgagaatccagaggtgctctatcgggctcccagaacaggcccagaaatattgttgcgcattgcagagaaagtccatagcccaggtgttgtgtcatcattgtcttctggcaatgcagtttgtaaagtcatggcctcagctgtatacttcttatcagcaacaatttttgaggacactggacgtcctgagttcaacttcttggaggattctaagtacagcttgctacaaaagatggctgcatattctggctttcatggtttcaatgatatggagccagaagatatattattcttattcccgaatattgaggaattagaatcactggattctatagtttacaacaagggagaaatagacatcatcccaagagtcaacatcagggatgcaacccaaaccagggtcactatctttaatgagcagaagaccctccggacatctccagagaagttggtgtcagacaagtggtttgggactcagaagagtaggataggcaaaacaaccttcctggctgaatgggaaaagctaaagaaaattgtaaagtggttggaagacactccagaagcaactctagctcacaccccactgaataaccatattcaagttaggaatttctttgctagaatggaaagcaagcctagaacagtcagaataacaggagctccagtaaagaagaggtcaggggttagtaagatagctatggttatccgtgacaatttctcccggatgggccatcttcgaggtgtagaagaccttgctggcttcactcgtagtgtgtcagctgaaattctcaagcactttctattctgtatactacaaggtccatacagtgagagctataagctacagctaatctacagagtcctaagctcagtgtcaaacgttgagataaaggaatcagatggtaagacaaaaaccaacttgattggaatccttcagagatttctagatggtgatcacgttgtccccataattgaagagatgggagccggaacagtgggtggattcatcaagagacaacaatctaaagttgtgcagaacaaagtggtctattatggagttgggatttggagaggcttcatggatggatatcaggtccatctagagatagaaaatgacataggacagcccccaaggcttaggaatgtcacaactaactgtcagagcagcccatgggacctgagtattccaataaggcaatgggcagaagacatgggggtcacaaacaaccaggattattcttctaaatctagcaggggggccagatattggatgcattcattcaggatgcaaggacctagcaagccatttggatgcccagtttatattattaagggtgatatgtcagatgtcatcagactgagaaaggaggaggtggagatgaaagtacggggctctactctcaacttgtacaccaagcaccattctcatcaggacctacacattctatcttacactgcatcagacaatgatctcagtccaggcattttcaagtcaatatcagatgagggggtggctcaagccctgcaattatttgagagggagccaagcaactgctgggtgagatgtgagtctgtagccccaaaatttatatcagccatccttgagatatgtgaggggaagagacagataaagggaattaacagaaccagactctcagagattgtgagaatttgttctgaatcttccctaagatcaaaagtcggatctatgttctcatttgtcgccaatgtcgaggaggcccatgatgttgattatgatgcgttaatggatctaatgatagaggatgccaagaacaatgcattcagtcatgttgttgactgcatagagttggatgttagtggcccttacgagatggagtcttttgatacatctgatgtcaatctctttgggccagcccattacaaggacatcagttcattatctatgattgctcatcccttaatggataagtttgttgattatgctatttctaagatggggagagcctcagttaggaaagttctagaaacaggtcggtgctccagcaaagactatgatttatcaaaggttctcttcagaactctacagagaccagaagaaagcattaggatagatgatctggaattatatgaggagacagatgtggcggatgacatgctaggctaagaccagaagagctctgtcaagggtcaagacaattctgcagatatccagcacagtggcggccgctcgaggaattctagatcccacgtcactattgtatactctatattatactctatgttatactctgtaatcctactcaataaacgtgtcacgcctgtgaaaccgtactaagtctcccgtgtcttcttatcaccatcaggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggt

SEQ ID NO: 26 - DNA, 7954 bp, artificial sequence
  vector
atgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacttttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcaattccctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctggggatctagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatctttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagctcctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgatcggatcccgggtaccgagctcggatccagtacccttcaccgacagagctcttctaaatgtatgttttattaacaattctaatctcggttctggtgtgtgaagcggttattagagtgtctctaagctccacaagagaagagacctgctttggtgactccactaacccagagatgattgaaggagcttgggattcactcagagaggaggagatgccggaggagctctcctgttctatatcaggcataagagaggttaagacctcaagccaggagttatacagggcattaaaagccatcattgctgctgatggcttgaacaacatcacctgccatggtaaggatcctgaggacaagatttccctcataaagggtcctcctcacaaaaagcgggtggggatagttcggtgtgagagacgaagagatgctaagcaaatagggagagaaaccatggcagggattgcaatgacagtccttccagccttagcagtttttgctttggcacctgttgtttttgctgaagacccccatctcagaaacagaccagggaaggggcacaactacattgacgggatgactcaggaggatgccacatgcaaacctgtgacatatgctggggcatgtagcagttttgatgtcttgcttgaaaagggaaaatttccccttttccagtcgtatgctcatcatagaactctactagaggcagttcacgacaccatcattgcaaaggctgatccacctagctgtgaccttctgagtgctcatgggaacccctgcatgaaagagaaactcgtgatgaagacacactgtccaaatgactaccagtcagctcattacctcaacaatgacgggaaaatggcttcagtcaagtgccctcctaagtatgagctcactgaggactgcaacttttgtaggcagatgacaggtgctagcctgaagaaggggtcttatcctctccaagacttgttttgtcagtcaagtgaggatgatggatcaaaattaaaaacaaaaatgaaaggggtctgcgaagtgggggttcaagcactcaaaaagtgtgatggccaactcagcactgcacatgaggttgtgccctttgcagtgtttaagaactcaaagaaggtttatcttgataagcttgaccttaagactgaggagaatctgctaccagactcatttgtctgtttcgagcataagggacagtacaaaggaacaatggactctggtcagactaagagggagctcaaaagctttgatatctctcagtgccccaagattggaggacatggtagtaagaagtgcactggggacgcagcattttgctctgcttatgagtgcactgctcagtacgccaatgcctattgttcacatgctaatgggtcagggattgtgcagatacaagtatcaggggtctggaagaagcctttatgtgtagggtatgagagagtggttgtgaagagagaactctctgccaagcccatccagagagttgagccttgcacaacttgtataaccaaatgtgagcctcatggattggttgtccgatcaacagggttcaagatatcatcagcagttgcttgtgctagcggagtttgcgtcacaggatcacagagtccttccaccgagattacactcaagtatccagggatatcccagtcttctgggggggacataggggttcacatggcacacgatgatcagtcagttagctccaaaatagtagctcactgccctccccaggacccgtgcttagtgcatgactgcatagtgtgtgctcatggcctgataaattaccagtgtcacactgctctcagtgcctttgttgttgtgtttgtattcagttctattgcaataatttgtttagctattctctatagggtgcttaagtgcctgaagattgccccaaggaaagttctgaatccactaatgtggatcacagccttcatcagatggatatataagaagatggttgccagagtggcagacaacattaatcaagtgaacagggaaataggatggatggaaggaggtcagttggttctagggaaccctgcccctattcctcgtcatgccccaatcccacgttatagcacatacctgatgttattattgattgtctcatatgcatcagcatgttcagaactgattcaggcaagctccagaatcaccacttgctctacagagggtgttaacaccaagtgtagactgtctggcacagcattgatcagagcagggtcagttggggcagaggcttgtttgatgttgaagggggtcaaggaagatcaaaccaagttcttaaagataaaaactgtctcaagtgagctatcatgcagggagggccagagttattggactgggtcctttagccctaaatgtttgagctcaaggagatgccaccttgtcggggaatgccatgtgaataggtgtctgtcttggagggacaatgaaacttcagcagagttttcatttgttggggaaagcacgaccatgcgagagaataagtgttttgagcaatgtggaggatgggggtgtgggtgtttcaatgtgaacccatcttgcttatttgtgcacacgtatctgcagtcagttagaaaagaggcccttagagtttttaactgtatcgactgggtgcataaactcactctagagatcacagactttgatggctctgtttcaacaatagacttgggagcatcatctagccgtttcacaaactggggttcagttagcctctcactggacgcagagggcatttcaggctcaaatagcttttctttcattgagagcccaggcaaagggtatgcaattgttgatgagccattctcagaaattcctcggcaagggttcttgggggagatcaggtgcaattcagagtcctcagtcctgagtgctcatgaatcatgccttagggcaccaaaccttatctcatacaagcccatgatagatcaattggagtgcacaacaaatctgattgatccctttgttgtctttgagaggggttctctgccacagacaaggaatgacaaaacctttgcagcttcaaaaggaaatagaggtgttcaagctttctctaagggctctgtacaagctgatctaactctgatgtttgacaattttgaggtggactttgtgggagcagccgtatcttgtgatgccgccttcttaaatttgacaggttgctattcttgcaatgcaggggccagggtctgcctgtctatcacatccacaggaactggatccctctctgcccacaataaggatgggtctctgcatatagtccttccatcagagaatggaacaaaagaccagtgtcagatactacacttcactgtgcctgaagtagaggaggagtttatgtactcttgtgatggagatgagcggcctctgttggtgaaggggaccctgatagccattgatccatttgatgataggcgggaagcagggggggaatcaacagttgtgaatccaaaatctggatcttggaatttctttgactggttttctggactcatgagttggtttggagggcctcttaaaactatactcctcatttgcctgtatgttgcattatcaattgggctctttttcctccttatatatcttggaagaacaggcctctctaaaatgtggcttgctgccactaagaaggcctcatagatcaggaagagctctgtcaagggtcaagacaattctgcagatatccagcacagtggcggccgctcgaggaattctagatcccacgtcactattgtatactctatattatactctatgttatactctgtaatcctactcaataaacgtgtcacgcctgtgaaaccgtactaagtctcccgtgtcttcttatcaccatcaggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggt

SEQ ID NO: 27 - DNA, 7954 bp, artificial sequence; identical to SEQ ID NO: 26
  vector

SEQ ID NO: 28 - DNA, 7954 bp, artificial sequence; identical to SEQ ID NO: 26
  vector

(19) United States
(12) Patent Application Publication
(10) Pub. No.: US 2011/0110976 A1
(43) Pub. Date: May 12, 2011

(54) RIFT VALLEY FEVER VIRUS-LIKE PARTICLES AND THEIR USE FOR IMMUNIZATION AND AS TEST SYSTEM

(75) Inventors: Friedemann Weber, Freiburg (DE); Matthias Habjan, Freiburg (DE); Martin Spiegel, Gottingen (DE); Nicola Penski, Freiburg (DE)

(73) Assignee: Universitaetsklinikum Freiburg, Freiburg (DE)

(21) Appl. No.: 12/997,370

(22) PCT Filed: Jun. 9, 2009
(86) PCT No.: PCT/EP09/57078

§ 371 Date: Jan. 5, 2011

(30) Foreign Application Priority Data
Jun. 10, 2008 (EP) 08010548.9

(51) Int. Cl. A61K 39/12; C12P 21/02; C07K 14/175; A61K 38/02; C12Q 1/02; G01N 33/68; A61P 37/04; A61P 31/14

(52) U.S. Cl. 424/204.1; 435/69.1; 530/350; 514/1.1; 435/29; 435/7.1

(57) ABSTRACT

Disclosed is a method for producing virus-like particles (VLP) that comprises the steps of (a) transfecting a mammalian cell line with several independent vectors, including (1) a vector containing at least a substantial part of the M gene from Rift Valley Fever Virus, (2) a vector containing at least a substantial part of the L gene from Rift Valley Fever Virus, (3) a vector containing at least a substantial part of the N gene from Rift Valley Fever Virus and (4) a vector containing a multicloning site flanked by the non-coding 5′- and 3′-ends of the L-, M- or S-segment of Rift Valley Fever Virus, and (b) culturing the transfected mammalian cell line under suitable conditions and obtaining the VLPs from the supernatant of the transfected cultured cell lines. The so-obtained VLPs can be used for vaccination and in methods for testing the antiviral activity of compounds.

Rift Valley fever Virus (RVFV) is a serious emerging pathogen affecting humans and livestock in sub-Saharan Africa, Egypt, Yemen, and Saudi Arabia. Since the first description of an outbreak in Kenya in 1931, there have been recurrent epidemics, killing thousands of animals, hundreds of humans, and causing significant economic losses. To animals, RVFV is mainly transmitted by mosquitoes, but humans are often infected by close contact with infected animals' material. A wide array of symptoms is connected with the human disease, ranging from an uncomplicated acute febrile illness to retinitis, hepatitis, meningoencephalitis, severe hemorrhagic disease, and death. The severity of RVFV zoonosis as well as the capability to cause major epidemics in livestock and humans have prompted authorities to list RVFV as a notifiable disease and a potential biological weapon.

RVFV belongs to the genus Phlebovirus, family Bunyaviridae. Bunyaviruses are enveloped and have a tri-segmented single-stranded RNA genome of negative or ambisense polarity, replicate in the cytoplasm, and bud into the Golgi apparatus. They encode five common structural proteins:

the viral polymerase (L) on the large (L) segment,
two glycoproteins (Gn and Gc) and the 78 kD protein on the medium (M) segment, and
the viral nucleocapsid protein (N) on the smallest (S) segment.

RVFV additionally expresses two nonstructural proteins, encoded on the M segment (termed NSm) and the S segment (termed NSs). These accessory proteins are dispensable for viral multiplication in cell culture, but play important roles for pathogenesis in vivo. In particular, the NSm and 78 kD proteins were found to enhance intrahost viral spread, whereas NSs is important to suppress the antiviral interferon system.

The general features of RVFV transcription and replication are similar to those of other negative-stranded RNA viruses. The viral genomic RNA (vRNA) segments contain untranslated regions (UTRs) on both the 3′ and the 5′ ends that serve as promoters for replication of the segment and transcription of the encoded reading frames. The vRNAs are encapsidated by N protein and associate with L protein both intracellularly and in the virion, and only these ribonucleoprotein particles (RNPs) are functional templates for mRNA synthesis and RNA replication by the viral polymerase.

After Gn/Gc-mediated entry into the cytoplasm, the negative-sense vRNAs of RVFV are transcribed into mRNAs by the RNP-associated L and N proteins (“primary transcription”). The products of these immediate early mRNAs then provide the machinery for replication of the genome and further mRNA synthesis (“secondary transcription”), and later drive the packaging of newly assembled RNPs into budding particles. A specific feature of bunyaviruses is that their mRNA synthesis is strictly dependent on host translation. Most likely, ongoing translation prevents premature termination of transcription by secondary structures building up on the nascent mRNA.

The infection cycle of bunyaviruses is strongly inhibited by the interferon-induced MxA protein of humans. The cytoplasmatically located MxA is capable of binding recombinant N protein of RVFV and several other bunyaviruses, and to sequester it to perinuclear complexes. It remained unclear, however, whether MxA solely cuts bunyaviruses off the supply of N protein, or whether it also affects assembled RNPs and inhibits their transcriptional activity.

Recently, reverse genetic systems to rescue infectious RVFV particles entirely from cloned cDNA plasmids have been developed (Ikegami, Journal of Virology [May 2005], pp 5606-5615; Gerrard et al., Virology 359, 459-65 [2007]). These systems allow the targeted manipulation of the viral genome and proved to be extremely useful to demonstrate the contribution of the NSs and the NSm proteins to viral pathogenesis and to identify transcriptional termination signals.

However, research on RVFV is hampered and restricted since the pathogen can only be handled under biosafety level 3 (BSL3) conditions. Therefore, it would be desirable to have tools at hand which allow to study aspects of RVFV infection cycle and the host response under non-BSL3 conditions.

WO 2007/104979 discloses a method for producing virus like particles for RVFV in a baculovirus system. Överby et al., J. Virol. (2006), pp. 10428-10435 describe the generation and analysis of VLPs of Uukuniemi virus as a system for studying Bunyaviral packaging and budding.

The systems disclosed in WO 2007/104979 and Överby et al. is not suitable for VLP mediated gene expression. The system of Överby et al. requires transfection of target cells with the polymeric constructs L and N prior to VLP infection. The RVFV VLPs of the present invention are capable of autonomous gene expression (primary transcription) without the need for additional manipulations of the target cells. Furthermore, it has not been shown that said VLPs can induce an antibody response and that they are suitable as vaccine. Concerning vaccination the article published by Överby et al. is not relevant since Uukuniemi virus is not pathogenic for animals or humans, respectively.

The establishment of a RVFV reverse genetics system which recapitulates the first steps of the replication cycle in a modular manner is disclosed. A minireplicon system was developed to measure RVFV polymerase activity. Additional expression of viral glycoproteins allowed efficient and helper-free packaging of minireplicon RNPs into virus-like particles (VLPs) which were released into the medium. By incubating naive cells with VLPs, the first steps of RVFV infection are reconstituted. Importantly, the system allowed to demonstrate that the L polymerase complexed with incoming RNPs is highly active in primary transcription and, if both L and the nucleoprotein N are additionally provided in trans, replication as well. The suitability of our modular approach was further validated by testing whether the antiviral protein MxA inhibits the primary and/or the secondary transcription of RVFV. The cytoplasmic human MxA which is encoded by chromosome 21 has antiviral activity against several viruses, in particular against Bunyaviruses.

The present invention discloses a method for producing virus-like particles whereby a mammalian cell line, preferably a human cell line, is transfected with several independent vectors. The cell lines are preferably derived from human origin since the virus-like particles are preferably used as vaccine which provides immunity against an infection of Rift Valley Fever Virus. The host cell is higher eukaryotic (for example, all mammals, including but not limited to mouse and human) and may be used for expression of the desired coding sequences when appropriate control sequences which are compatible with the designated host are used.

In a particularly preferred embodiment of the present invention the yield of the VLPs is increased by inhibiting the antivirally active kinase PKR. The kinase PKR can be inhibited by adding a potent PKR inhibitor, in particular a dominant negative mutant of PKR, for example PKR delta E7.

Mammalian cell lines available as hosts for expression of cloned genes are known in the art and include many immortalized cell lines available from the American Type Culture Collection (ATCC), such as HEp-2, CHO cells, Vero cells, baby hamster kidney (BHK) cells and COS cells or BSR-T7/5 cells, to name a few. Suitable promoters are also known in the art and include viral promoters such as that from SV40, Rous sarcoma virus (RSV), adenovirus (ADV), bovine papilloma virus (BPV), and cytomegalovirus (CMV).

Mammalian cells may also require terminator sequences, poly A addition sequences, enhancer sequences which increase expression, or sequences which cause amplification of the gene. These sequences are known in the art.

Preferred cell lines are selected from the group consisting of cell lines having the well-known scientific designations HEK 293, 293T, Vero, HeLa, COS-1, A549, Huh-7, Huh-7.5, Hep2G, Jurkat and/or BSR-T 7/5.

The advantage of the present method is that the cell lines do not contain infections with Rift Valley Fever Virus or inactivated RVFV virus at any time during the production of VLPs. Therefore, there is no risk that by recombinational events viable and infectious dangerous viruses emerge. The genes M, L and N, respectively, are cloned into a vector which can transiently express the open reading frames of the M, L and N genes. It is not necessary that the complete M, L and/or N gene from RVFV is contained within the vectors. It is sufficient if the sequence coding for M, L and N are present in a form which shows a homology of at least 80%, preferably at least 90% to the gene calculated over the whole length of the gene. According to the present invention at least a substantial part of the gene coding for M, L and/or N is present in the vector. The term “at least a substantial part of the gene” means that preferably at least 90% and more preferred at least 95% of the wild type gene are present.

Furthermore the cell line is transfected with a vector which is designated in the present application as a minireplicon plasmid. The minireplicon plasmid contains a multicloning site flanked by the non-coding 5′- and 3′-ends of the L, M or S segment of Rift Valley Fever Virus. The virus like RNA encoded by the minireplicon plasmid is not self-replicating but requires the coexpression of N and L genes which lead to transcription and replication.

In a particularly preferred embodiment of the present invention the autonomous transcription by the VLPs is enhanced by coexpressing the viral N gene (which is a transcriptional enhancer) with a gene of interest, e.g. a reporter, on a single minireplicon. Coexpression is achieved by using a minireplicon resembling the viral S segment, which encodes two genes in a so-called ambisense manner.

The advantage of this minireplicon is that an artificial gene segment can be inserted into the virus-like particles. Moreover, by packaging a suitable foreign gene into the VLP with the help of the minireplicon system it is possible to enhance the immunological activity and to improve the efficacy of VLPs in the immunization of patients and/or animals.

The inserted gene may be a gene obtained from RVFV like for example a gene coding for the Gn and/or Gc protein or the nucleoprotein N or the polymerase L. Expression of the N gene has already been achieved and proved to strongly enhance the immunogenicity of the RVFV-VLPs (see example 7). Alternatively the gene may code for a protein which stimulates the immune response. It may for example code for a ligand to which a receptor of immune cells may bind or alternatively interferons like IFN-beta, immuncytokines like IL-2, CXCL-10. In the experiments of the present application the minireplicon contained a luciferase gene obtained from Renilla reniformis as model system in order to show that the gene contained within the minireplicon is expressed. In the vaccination experiments VLPs comprising an artificial gene segment preferably encoding the RVFV N gene fragment were used.

In a preferred embodiment the minireplicon is transcribed with the help of an RNA-polymerase I promoter which may be obtained from human origin and/or mouse. Alternatively the use of a T7 polymerase promoter is also possible.

The invention relates in another embodiment to virus-like particles (VLP) obtainable by the method of the present invention. Preferably such VLPs are used in pharmaceutical compositions as vaccine against an infection of RVFV. The VLPs of the present invention have several advantages. Since the VLPs are produced in mammalian, preferably human cell lines they have a glycosylation pattern on the protein which is specific for human. Therefore, there is no risk that undesired immunological reactions may occur. Such risk can, however, not be excluded when products are used which are produced in other organisms like plant cells and/or insect cells. The folding of the glycoproteins of the VLPs according to the present invention corresponds exactly to the usual folding of human proteins. Therefore, it can be concluded that immunological reactions of the immunized individual are specifically directed against proteins contained within the VLPs obtained from Rift Valley Fever Virus like N, L, G proteins.

In an alternative embodiment the virus-like particles are used for the vaccination of animals, in particular sheep and/or cattle. Since the virus can cause substantial losses in agriculture there is a demand to provide an efficient system for vaccination of animals like cows, sheep, horses, goats or camels. For preparing suitable VLPs it is preferred to use cell lines of the same species to be vaccinated. For the vaccination of cows, preferably bovine cell lines are used.

An important advantage of the VLPs of the present application is that their proteins correspond exactly to the proteins derived from the virus without, however, the risk that an attenuated virus used for immunization may be reactivated by undesired recombination events resulting in undesired side-effects.

A further advantage of the present invention is the ability to autonomously transcribe any gene encoded on the VLP minireplicon RNA. This so-called primary transcription allows to express the gene of interest in infected cells without any further manipulation such as co-transfection of L and N gene (as shown for example in FIG. 5B).

The virus-like particles of the present invention can be used in a method for testing whether an agent has antiviral activity against Rift Valley Fever Virus. This test method is a kind of screening method wherein VLPs prepared according to the teaching of the present application are brought into contact with a mammalian cell line which can be infected by the VLPs. Additionally, an agent which is supposed to have antiviral activity is added to the test sample. In a comparative test the same amount of the VLPs and cells of the same cell line are brought together whereby the agent to be tested is replaced by a suitable control.

The control can be either the corresponding amount of buffer without any agent or it can be a corresponding compound which does not have an antiviral activity. One example of the agent to be tested is the antiviral human protein MxA. In the examples (see FIG. 6) the human protein MxA has been tested in an active form compared with an inactive form.

Furthermore, the testing method is suitable for determining the activity of neutralizing antibodies having activity against RVFV or for testing any chemically synthesized compound or compounds derived from naturally occurring substances which are supposed to have antiviral activity. The advantage of the test method is that it can be performed under relatively low security conditions, since no viable virus is used. Furthermore, the test can be easily automated, standardized and a high throughput of compounds to be screened is possible.

A screening test for agents having antiviral activity can be performed in the following manner. A suitable cell line (e.g. BHK cells) were seeded in equal amounts into plates having 96 wells. After growth of the cells for one day, about 50 μl of a VLP solution containing about 10⁴ active VLPs are dispensed in each well and the cells are incubated for one hour. Thereafter to each well an aliquot of about 50 μl medium is added whereby the test solution contains the agent to be tested and the control does not contain the agent to be tested. After incubation over night the cells are suspended in 20 μl passive lysis buffer and the Renilla activity is measured. By using microtiter plates having for example 96 wells it is easy to perform the test in an automated manner by using suitable laboratory automated devices.

The present invention is shown in the enclosed Figures and preferred embodiments are described in the examples.

### DESCRIPTION OF FIGURES

FIG. 1: Minireplicon system for RVFV.

Human 293T cells were transfected with expression plasmids for the nucleoprotein N and the viral polymerase L, together with a minireplicon construct containing the Ren-Luc reporter gene in antisense flanked by the untranslated regions of the M segment (vM-Ren). As a negative control the L expression plasmid was omitted. An FF-Luc plasmid was cotransfected to control for background expression. Cell lysates were assayed 24 h post transfection for Ren-Luc and FF-Luc activity. Luciferase counts were normalized to the experiment without L, numbers on top of each column indicating fold induction. Data from a representative experiment are shown.

FIG. 2: Formation of RVFV VLPs.

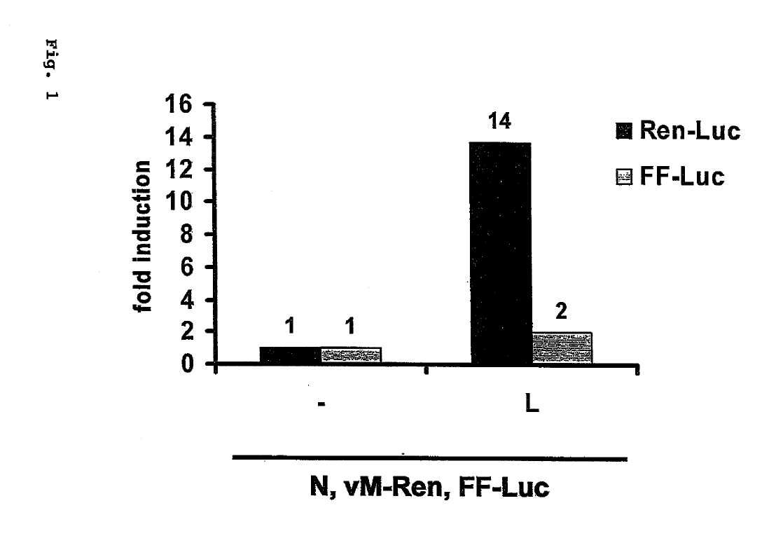

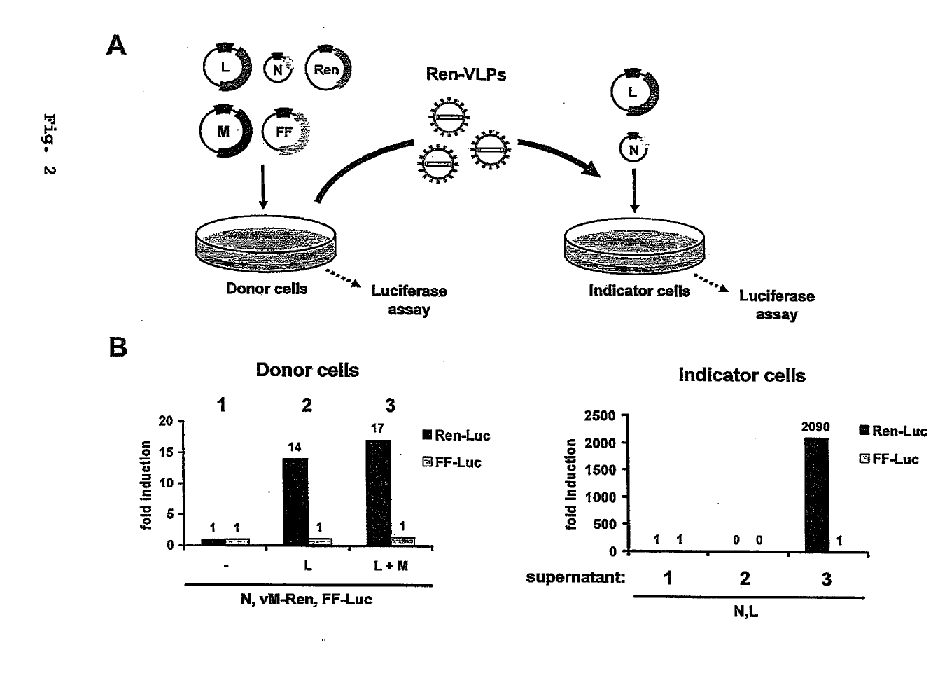

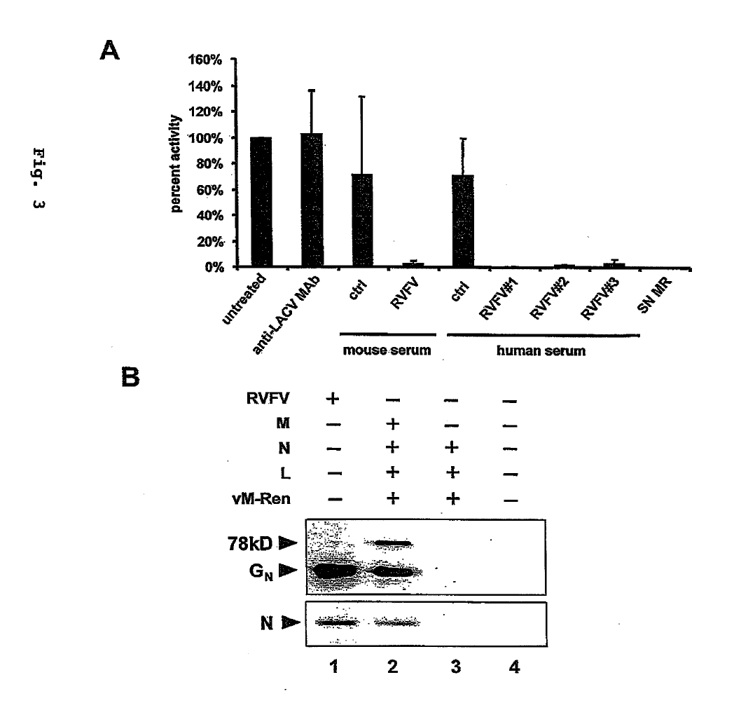

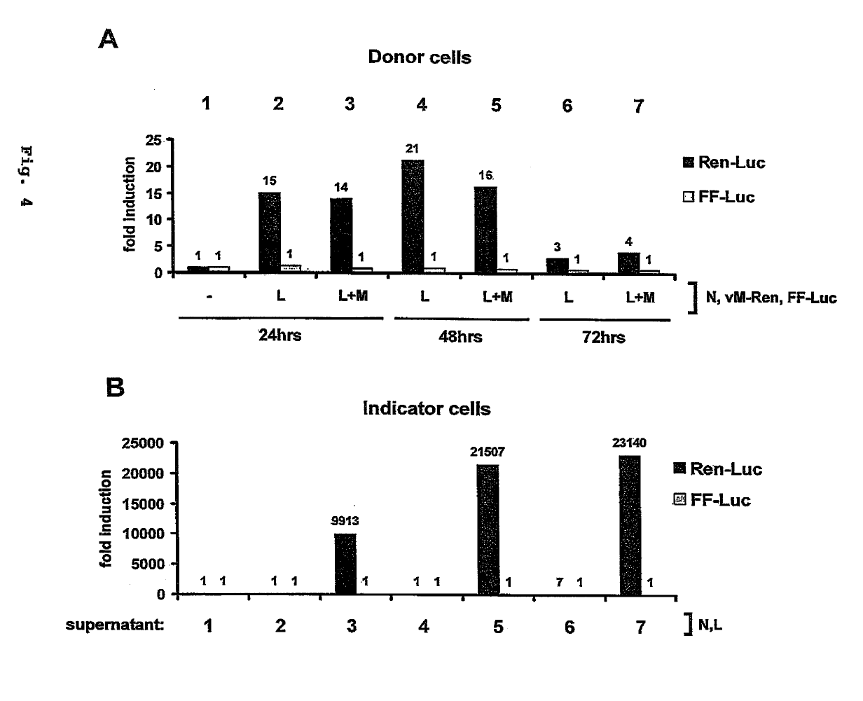

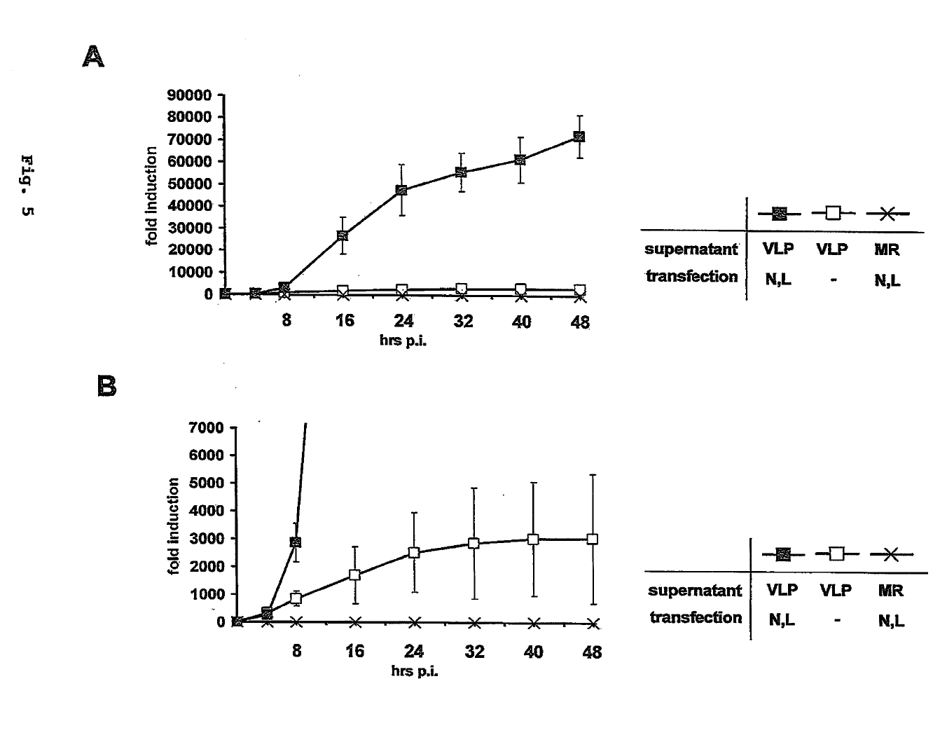

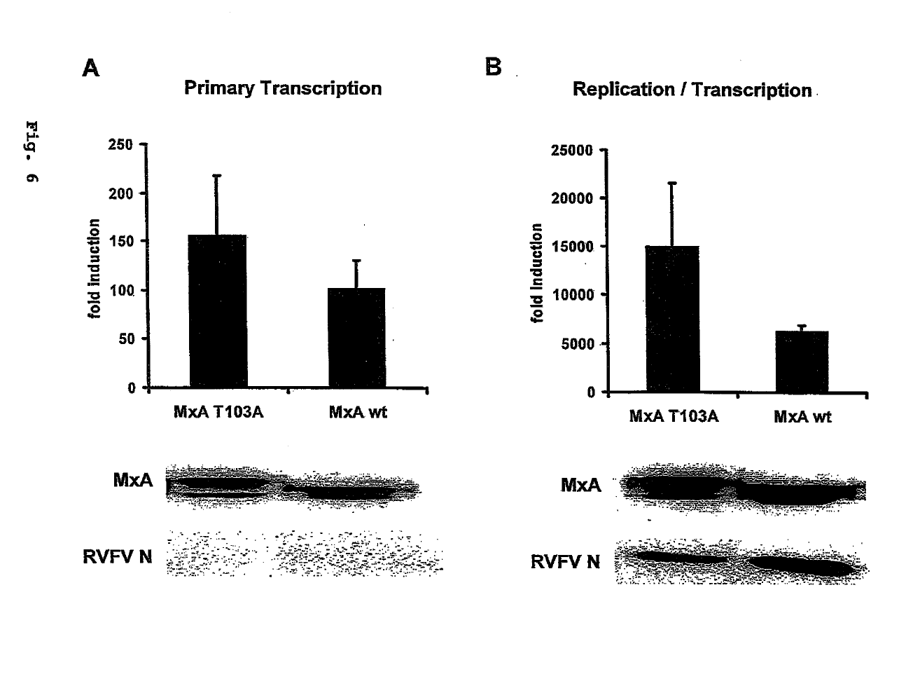

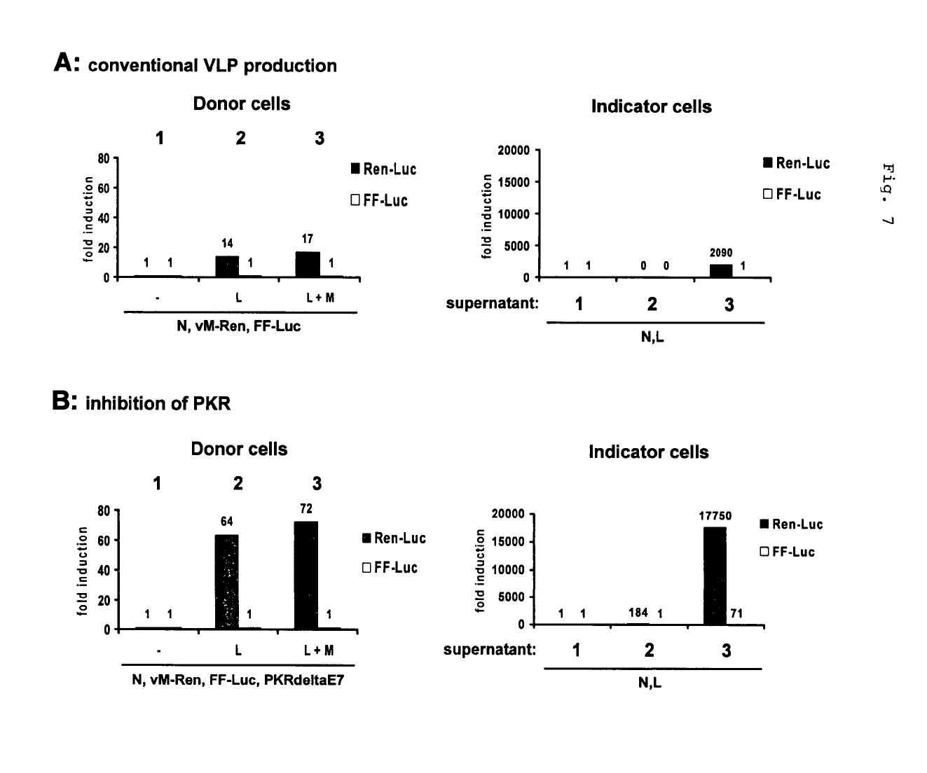

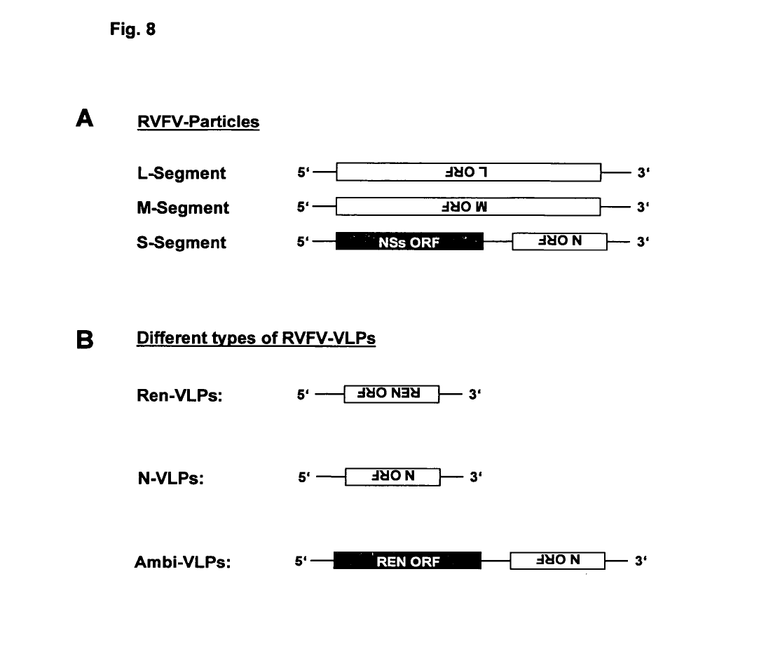

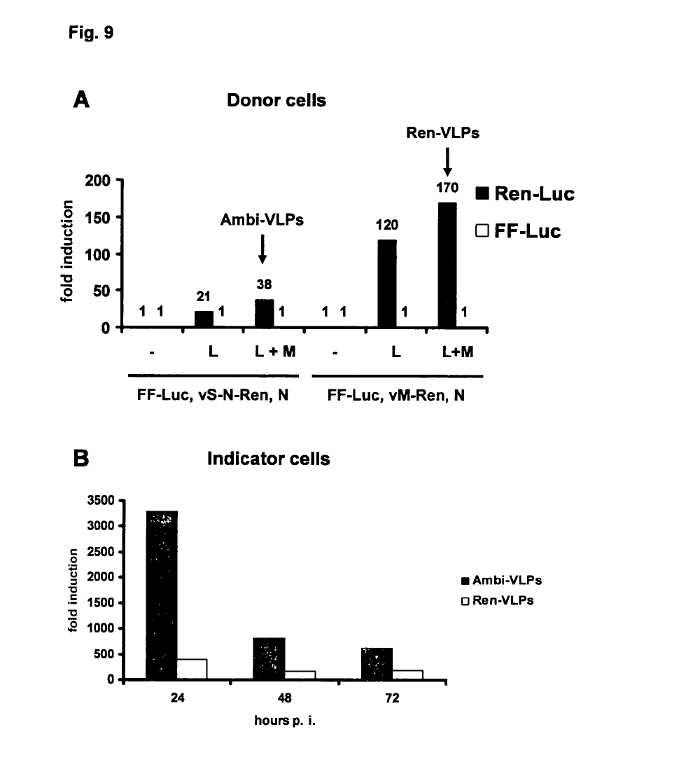

(A) Outline of the procedure used for generation of VLPs. Donor cells were transfected with expression plasmids for N, L and the viral glycoproteins (M), together with the reporter minireplicon construct vM-Ren. Release of VLPs into the supernatant of donor cells was detected by transfer of supernatants on indicator cells expressing both N and L. (B) Luciferase activities in donor and indicator cells. 293T cells (donor cells) were transfected with minireplicon system plasmids alone as described in FIG. 1 (left panel, columns 1 and 2), or together with an M segment expression plasmid (left panel, column 3). Supernatants were harvested 24 h later and used to infect BSR-T7/5 indicator cells (right panel). FF-Luc and Ren-Luc activities in donor cells were measured upon removal of supernatants, and indicator cells were analyzed 24 h post infection. Luciferase counts were normalized to the experiment where L has been omitted, and numbers on top of each column display fold induction. Data from a representative experiment are shown.

FIG. 3: VLPs resemble authentic RVFV particles.

(A) Neutralisation of VLPs by RVFV-specific antisera. VLP-containing supernatants were incubated with 5 μl of mouse or human antisera specific for RVFV for 1 hour at 37° C. As controls, 5 μl of a non-seroconverted mouse and human (ctrl) or of a monoclonal antibody against La Crosse bunyavirus glycoprotein. Gc were used. Cells were then infected with antibody-treated or untreated VLP preparations, or the supernatant from minireplicon-expressing cells (SN MR). Ren-Luc activity was measured 24 hours later and is shown as percent activity of untreated VLPs. Error bars show standard deviations from three independent experiments. (B). Western blot analysis of RVF virus particles and VLPs. Concentrated supernatants from RVFV-infected cells (lane 1) and 293T cells transfected with different plasmid combinations (lane 2 to 4) were analyzed by Western blotting using a monoclonal antibody specific for RVFV Gn and 78 kD protein (upper panel), and an antibody against the nucleoprotein N (lower panel).

FIG. 4: Optimisation of VLP production.

VLPs were generated as described in FIG. 2, and VLP release from donor cells was followed over the course of three days. Supernatants from parallel dishes were harvested at the timepoints indicated and used to infect indicator cells expressing N and L. (A) Ren-Luc and FF-Luc activities in donor cells were measured upon removal of supernatant. (B) Analysis of luciferase activities in indicator cells was performed 24 h post-infection. Luciferase counts were normalised to the experiment where L has been omitted. Data from a representative experiment are shown, numbers on top of each column displaying fold induction.

FIG. 5: Primary and secondary transcription in VLP-infected cells.

(A). BSR-T7/5 cells were either mock transfected (open squares), or transfected with N and L expression plasmids (filled squares) prior to infection with VLPs harbouring. Ren-Luc RNPs. As a negative control, cells transfected with N and L plasmids were infected with supernatants from minireplicon-expressing donor cells (crosses). Ren-Luc activities in VLP-infected cells were then measured at different timepoints over the course of 48 h. (B). The same data is shown as in A, but with the y-axis adjusted to visualise activity of VLPs in naive cells. Error bars show standard deviations from three independent experiments.

FIG. 6: Inhibition of both RVFV primary and secondary transcription by human MxA.

The influence of human MxA on RVFV primary transcription was analyzed by transfecting BSR-T7/5 cells with 1.5 μg of an expression construct for wt MxA (A). To study the effect of MxA on RVFV replication and secondary transcription, the same amount of plasmid for wt MxA was cotransfected together with 0.5 μg of each N and L (B). The inactive MxA-mutant T103A was used as a negative control. Twenty-four hours post transfection, cells were infected with VLPs harbouring Ren-Luc RNPs. Renilla luciferase activities in cell lysates were measured 24 hours later. Equal amounts of these lysates were used for Western Blot analysis to confirm expression of both MxA and RVFV N. Data are shown from three independent experiments performed in parallel, error bars indicating standard deviations.

FIG. 7: Increasing yield of VLPs.

FIG. 7 demonstrates the increasing of VLP yields by co-expression of the PKR-Inhibitor PKRdeltaE7. FIG. 7 (A) Donor cells (left panels) were either conventionally transfected with VLP constructs (column 3), or (B) additionally transfected with 0.5 μg of the expression construct pl.18-PKRdeltaE7. As specificity control was the M-expression construct (coding for the glycoproteins) omitted (column 2), as negative control we omitted the polymerase L as well as M. (column 1). Supernatants of donor cells were harvested at 48 h post-transfection and cells were lysed for luciferase assays. Indicator cells were transfected with N- and L-expression constructs 16 h before infecting them with 250 μl VLP-containing donor cell supernatants. Luciferase activities of the indicator cells taken 24 h after VLP infection show that—as expected—only the full set of VLP constructs (column 3) leads to production of VLPs. Addition of pl.18-PKRdeltaE7 into the VLP-transfection mix results in a ca. 10-fold increase of VLP activity (compare column 3 of indicator cells in A and B).

FIG. 8: Coding strategies.

FIG. 8 demonstrates the coding strategies of RVFV and RVFV-VLPs. Gene segments of RVFV (A) and genome segments of the different types of RVFV-VLPs (B). Note that Ren-VLPs and N-VLPs are based on the viral M segment, whereas the ambisense VLPs are based on the S segment. The Ren-ORF of the Ambi-VLPs can be replaced by any gene of interest.

FIG. 9: Enhancing autonomous VLP transcription by using ambisense-minireplicons

FIG. 9 demonstrates the comparison of ambisense-VLPs with conventional (Ren-) VLPs. (A) For production of the VLPs, 293T cells (donor cells) were transfected in 6-well dishes with 0.5 μg pl.18-N, 0.5 μg minireplicon construct (pHH21-vS-N-Ren for Ambi-VLPs and pHH21-vM-Ren for Ren-VLPs), 0.1 μg pGL3 ctrl, 0.5 μg pl.18-L, and 0.5 μg pl.18-M. 48 h later supernatants were harvested and donor cells lysed. Relative reporter activities of lysates are shown as columns. The negative control (omission of the L construct from the transfection mix) was set as 1. (B) Autonomous VLP activities. BHK indicator cells in 12-well dishes were incubated with 250 μl VLP supernatant (see A: L+M) lysed at the indicated time points, and the Ren-activities were measured.

### EXAMPLE 1

### Construction of Plasmids

All plasmids were generated using standard molecular cloning techniques and confirmed by DNA sequencing. PCR was carried out with AccuPrime Pfx DNA Polymerase (Invitrogen; for cloning purposes) or Taq DNA Polymerase (Eppendorf; for diagnostic purposes and addition of single adenosines for TA cloning). TA cloning was done using the pcDNA3.1N5-His TOPO TA Expression Kit (Invitrogen) according to the manufacturers instructions. The preparation of RVFV first-strand cDNA was performed as described previously for La Crosse virus (Blakqori et al., J. Gen. Virol. 84 (2003), pp 1207-1214).

Viral genes were amplified from the first-strand cDNA using specific primers as shown in Table 1.

TABLE 1
DNA oligonucleotide primers

| | SEQ | | Segment/ | Nucleo- |
|---|---|---|---|---|
| Name | ID NO. | Sequence (5′ to 3′)^{a} | Gene | tides^{b,c} |
| RVFV_SapI_L1for | 1 | GACAGAGCTCTTCATCATGGATTCTATATTATCAAAACAGCTG | L gene | 16-45 |
| RVFV_L1rev | 2 | TGGAGGCATCCATTGCTGC | L gene | 1959-1942 |
| RVFV_L2for | 3 | GCTGGGCCTTTGATCTCTC | L gene | 1727-1745 |
| RVFV_L2rev | 4 | CTCCCGATGACCATCCAG | L gene | 3159-3142 |
| RVFV_L3for | 5 | GAGGAAAGAATTGTTCAATCGG | L gene | 2818-2839 |
| RVFV_L3rev | 6 | AATGGTCACGGATAACCATAGC | L gene | 4867-4846 |
| RVFV_L4 for | 7 | AAGCAACTCTAGCTCACACCCC | L gene | 4712-4733 |
| RVFV_L4rev_SapI | 8 | GACAGAGCTCTTCTGGTCTTAGCCTAGCATGTCATC | L gene | 6302-6280 |
| RVFV_SapI_M1for | 9 | GACAGAGCTCTTCTAAATGTATGTTTTATTAACAATTCTAATCTCG | M gene | 18-50 |
| RVFV_M1rev | 10 | TCAAACAAGCCTCTGCCC | M gene | 2217-2200 |
| RVFV_M2for | 11 | GTGAACAGGGAAATAGGATGG | M gene | 1956-1976 |
| RVFV_M2rev_SapI | 12 | GACAGAGCTCTTCCTGATCTATGAGGCCTTCTTAGTG | M gene | 3619-3596 |
| RVFV_SapI_Sfor | 13 | GACAGAGCTCTTCATAATGGACAACTATCAAGAGCTTGC | N gene | 36-61 |
| RVFV_C13_Srev_SapI | 14 | GACAGAGCTCTTCCAAGCAGCAAAAGAGGACATTTC | N gene | 1062-1040 |
| RVFV_cMPro1for | 15 | GACAGACGTCTCCTATAGACACAAAGACGGTGCATTAAATGAAGAGCG | M UTR | 1-//-3634 |
| | | GTACCGCTCTTCATCAGTACGTGTAAAAGCAA | | |
| RVFV_cMPro2rev | 16 | TGACCTATCTGTACAATACTTACATAATTAGGTTTGCTTATGTCTACT | M UTR | 3675-3754 |
| | | TATTTCAACATATTGCTTTTACACGTACTGAT | | |
| RVFV_cMPro3for | 17 | AGTATTGTACAGATAGGTCAAATTATTGGAATATCCAAGCTTAGAAAC | M UTR | 3814-3735 |
| | | TTATGCAATAATACTTTAGATGTAAGCTTAGT | | |
| RVFV_cMPro4rev | 18 | ACTAGAATATTCACAAGCACTTGAGACTGCTGCTGCCTCACCCCACCA | M UTR | 3795-3874 |
| | | CCCCAAATTACAACTAAGCTTACATCTAAAGT | | |
| RVFV_cMPro5for | 19 | GTGCTTGTGAATATTCTAGTTGGCGTAATCGTCTTTTGCCAGATTAGC | M UTR | 3885-3855 |
| | | TGGGAATTAAACTAACTCTTTGAAGTTGCACC | | |
| RVFV_cMPro6rev | 20 | TCTGTCCGTCTCCACCCACACAAAGACCGGTGCAACTTCAAAGAGTTA | — | — |
| RVFV_vMPro_pHH21for | 21 | GACAGACGTCTCCTATTACACAAAGACCGGTGCAACTTC | — | — |
| RVFV_vMPro_pHH21rev | 22 | GACAGACGTCTCCGGGACACAAAGACGGTGCATTAA | — | — |
| RVFV_MRen_SapIfor | 23 | GACAGAGCTCTTCAAAATGACTTCGAAAGTTTATGATCCAG | — | — |
| RVFV_MRen_SapIrev | 24 | GACAGAGCTCTTCTTGACTTATTGTTCATTTTTGAGAACTCGC | — | — |

^{a}nucleotides corresponding to RVFV sequences are in boldface letters
^{b}nucleotide numbers according to the database entries DQ375403 (L segment), DQ380206 (M segment), and
DQ380151 (S segment)
^{c}hybridization sites for reverse primers are indicated by reverse order numbering

The constructs pl.18_RVFV_L (SEQ ID NO:25), pl.18_RVFV_M (SEQ ID NO:26) and pl.18-RVFV_N (SEQ ID NO:27) contained the appropriate coding sequences subcloned into the eukaryotic high-level expression plasmid pl.18 (kindly provided by Jim Robertson, National Institute for Biological Standards and Control, Hertfordshire, UK). The minireplicon pHH21-vMRen (SEQ ID NO:28) was also constructed.

For construction of pl.18_RVFV_L, the L segment coding region was assembled in a stepwise manner from four overlapping cDNA fragments. These fragments were generated by PCR using primer pairs RVFV_Sapl_L1for/RVFV_L1rev, RVFV_L2for/RVFV_L2rev, RVFV_L3for/RVFV_L3rev, and RVFV_L4for/RVFV_L4rev_Sapl. The PCR fragments were individually TA-cloned into the pcDNA3.1-TOPO Vector (Invitrogen), giving rise to plasmids pcDNA3.1_RVFV_L1, pcDNA3.1_RVFV_L2, pcDNA3.1_RVFV_L3, and pcDNA3.1_RVFV_L4. Then, the insert of pcDNA3.1_RVFV_L2 was cut out with EcoRI and Xhol and cloned 3′ of the L1 insert into the EcoRI/Xhol-digested pcDNA3.1_RVFV_L1. The resulting plasmid was named pcDNA3.1_RVFV_L1+L2.

In parallel, fragment L4 was subcloned into pl.18 using the BamHI and Xhol restriction sites. The resulting plasmid, pl.18-L4 was then digested with Sacl and EcoRI. The gel-purified L4 fragment flanked by Sacl/EcoRI restriction sites at the 5′ and 3′ end, respectively, was then subcloned together with the BamHI/Sacl-digested L3 fragment (cut out of pcDNA3.1_RVFV_L3) into the BamHI/EcoRI-digested pl.18 vector. The resulting construct, pl.18_L3+L4, was then reopened with BamHI and Apal and the joined with the L1+L2 fragment cut out of pcDNA3.1_RVFV_L1+L2, again using BamHI/Apal. The amino acid sequence of the full-length insert in pl.18-RVFV L corresponds to the database entry DQ375403.

For construction of pl.18_RVFV_M two overlapping fragments were generated using primer pairs RVFV_Sapl_m1for/RVFV_m1rev and RVFV_m2for/RVFV_m2rev_Sapl. The two fragments were TA-cloned to generate plasmids pcDNA3.1_RVFV_M1 and pcDNA3.1_RVFV_M2, respectively. To obtain pl.18_RVFV_M, both fragments were finally cloned into the pl.18 vector in a three-way ligation reaction, where an internal DraIII-site served for joining of M1 and M2, and Kpnl and Xhol restriction sites were used for ligation into pl.18. The coding sequence of the insert corresponds to the database entry DQ380206, with the difference of one amino-acid substitution from leucine to glutamine at position 232.

Plasmid pl.18_RVFV_N was constructed by amplification of first-strand cDNA using PCR primers RVFV_Sapl_Sfor and RVFV_C13_Srev_Sapl. Subcloning of the resulting fragment into the TOPO vector first gave rise to plasmid pcDNA3.1_RVFV_N, from which the fragment was then cloned into the pl.18 vector using Kpnl/Xhol sites. The sequence of the insert corresponds to database entry DQ380151.

The reporter plasmid pHH21_RVFV_vMRen contains the Renilla luciferase gene (Ren-Luc) in antisense orientation, flanked by the 3′ and 5′ genomic untranslated regions (UTRs) of the RVFV M segment. This reporter plasmid was generated in a three-step manner. First, the M UTRs separated by a linker sequence containing two flanking Sapl restriction sites and one central Kpnl site were constructed from overlapping oligonucleotides RVFV_cMPro1for, RVFV_cMPro2rev, RVFV_cMPro3for, RVFV_cMPro4rev, RVFV_cMPro5for, RVFV_cMPro6rev as described previously (XXX). The resulting PCR fragment was TA-cloned to obtain pcDNA3.1_RVFV_MPro. Then, the UTR sequences including the linker were reamplified using primer pair RVFV_vMPro_pHH21for/RVFV_vMPro_pHH21rev and cloned into the pHH21 backbone vector via primer-encoded Esp3I sites. This gave rise to precursor plasmid pHH21_RVFV_vMPro. In a last step, the Ren-Luc gene was amplified from plasmid pRL-SV40 (Promega) using primers RVFV_MRen_Saplfor and RVFV_MRen_Saplrev, and inserted into pHH21_RVFV_vMPro using Sapl restriction sites, resulting in plasmid pHH21_RVFV_vMRen.

Plasmid pGL3-control (Promega) expresses firefly luciferase (FF-Luc) under control of a mammalian RNA polymerase II promoter.

Plasmid constructs encoding human MxA and the GTPase-deficient mutant T103A have been described previously [Ponten et al., J. of Virology 71:2591-9 (1997)]. For improved expression in mammalian cells we cloned the corresponding coding sequences into pl.18 via BamHI/EcoRI restriction sites, giving rise to plasmids pl.18-MxA and pl.18-HA-MxA (T103A).

### EXAMPLE 2

### RVFV Minireplicon System

Geneticin G418 (Biochrom. AG) was dissolved in H₂O to 100 mg/ml and used at a concentration of 1 mg/ml cell culture medium. BSR-T7/5 cells, Vero cells and 293T cells were cultivated in Dulbecco's modified Eagle's medium (DMEM) supplemented with 10% FCS.

Subconfluent monolayers of 293T cells in 12-well plates were transfected with 0.4 μg each pl.18_RVFV_N and pl.18_RVFV_L, 0.25 μg pHH21_RVFV_vMRen, and 0.1 μg pGL3-control using 3.5 μl Fugene HD transfection reagent (Roche) in 100 μl serum free medium (OptiMEM; Invitrogen). In some experiments, specific expression plasmids were omitted from the transfection mix. After transfection, cells were incubated for the indicated times and lysed in 100 μl Dual Luciferase Passive Lysis Buffer (Promega). An aliquot of 20 μl of the lysate was assayed for FF-Luc and Ren-Luc activities as described by the manufacturer (Promega).

Minireplicon systems are useful tools to investigate viral polymerase activity and promoter sequences. For bunyaviruses, they usually consist of a reporter gene flanked by viral 5′ and 3′ UTRs, and recombinant L and N proteins. The reporter construct is transfected into mammalian cells along with the L and N expression constructs and will be transcribed intracellularly into a minireplicon resembling a viral gene segment. The products of the expression constructs faithfully encapsidate, transcribe and replicate the minireplicon, thus resulting in reporter activity. For RVFV, minireplicon systems have been established which are based on the coexpressed RNA polymerase of the bacteriophage T7.

We disclose a T7-independent version in which the expression constructs were under control of a promoter for the cellular RNA polymerase II (RNAP II), and the minireplicon was transcribed intracellularly by RNAP I. The L and N genes were cloned into the high expression vector pl.18, and a minireplicon consisting of the antisense gene for the Renilla Luciferase (Ren-Luc) flanked by RVFV M segment UTRs was cloned into the RNAP I vector pHH21. Human 293T cells were transfected with these plasmids, and minireplicon activity was assessed after overnight incubation by measuring Ren-Luc activity of cell extracts.

FIG. 1 shows that coexpression of L, N and the minireplicon strongly upregulated the Ren-Luc activity (black bars), whereas omission of the L construct resulted in low activity. As the minireplicon plasmid was designed to produce negative sense RNA, a strong Ren-Luc activity clearly indicates successful packaging into RNPs and transcription by L and N. Moreover, the internal control consisting of the RNAP II-driven gene for the firefly luciferase (FF-Luc) was independent of the full set of RVFV plasmids (grey bars), as expected. Thus, our RVFV minireplicon system which is entirely based on the activity of cellular RNA polymerases was able to efficiently reconstitute recombinant RVFV RNPs.

### EXAMPLE 3

### Preparation of Virus-Like Particles

Subconfluent monolayers of 293T cells in 6-well plates (donor cells) were transfected with 0.5 μg of plasmids pl.18_RVFV_N, pl.18_RVFV_L, pl.18_RVFV_M, pHH21_RVFV_vMRen, and with 0.1 μg of pGL3-control using 6.3 μl Fugene HD transfection reagent. At the indicated timepoints supernatants were harvested and donor cells were lysed with 200 μl Passive Lysis Buffer to measure luciferase activities as described above. The supernatants were frozen for at least one hour at −20° C., treated with 1 μl/ml Benzonase (Novagen) at 37° C. for three hours, and centrifuged at 12.000×g for 5 min to remove cellular debris. Aliquots of the treated supernatants were then used to infect infect new cells (indicator cells). In some experiments, indicator cells were transfected with L and N expression plasmids 16 h prior to the transfer of supernatant to support replication and transcription of RNPs.

For two bunyaviruses, Bunyamwera virus and Uukuniemi virus, VLP systems consisting of minireplicon RNPs packaged by viral glycoproteins were established and have been proven useful for the analysis of particle assembly and RNP packaging. To achieve this for RVFV, we cloned the reading frame of the viral M segment (encoding Gn, Gc, NSm, and the 78 kD protein) into the RNAP II expression vector pl.18 (see Example 1). Immunofluorescense analyses using specific antisera confirmed expression of RVFV glycoproteins and their transport to the plasma membrane.

To generate VLPs, the M expression construct was cotransfected with the minireplicon system plasmids into 293T cells (donor cells). Twenty-four hours later, supernatants were harvested and used to infect BSR-T7/5 cells expressing RVFV L and N (indicator cells). An outline of the experiment is provided in FIG. 2 A.

Two additional measures were taken to minimize unspecific reporter activities in indicator cells caused by carryover transfection. Firstly, any residual plasmids were destroyed by extensive treatment of VLP supernatants with DNase. Secondly, the species-specificity of the human RNAP I-driven minireplicon construct excluded background expression in the non-human indicator cells. Moreover, RNAP II-driven coexpression of FF-Luc in donor cells served as a specificity control, because the FF-Luc RNA lacks any RVFV sequences and should therefore not be packaged into VLPs.

Reporter assays shown in FIG. 2 B (left panel) demonstrate again the strong minireplicon activity mediated by L and N. Coexpression of the M segment-encoded glycoproteins had no detrimental effects.

Importantly, after transfer of supernatants to indicator cells, only the sample which had received the M expression construct in addition to the minireplicon system displayed a strong Ren-Luc activity. FF-Luc activity, by contrast, was neglectable in indicator cells. These data implicate that coexpression of the M segment-encoded genes results in efficient and specific packaging of minireplicon RNPs into VLPs, which are subsequently released into the medium, able to infect new cells.

We further analysed VLP characteristics by using neutralising antisera. VLP-containing supernatants were first incubated with different antisera derived from RVFV-infected mice or humans, and then used to infect indicator cells expressing RVFV L and N.

Proteins were separated by SDS-PAGE and transferred to a PVDF membrane (Amersham), followed by incubation in saturation buffer (PBS containing 10% non-fat dry milk and 0.05% Tween). The membrane was first incubated for one hour with primary antibodies, washed three times with 0.05% PBS-Tween, followed by incubation with a horseradish-peroxidase (HRP)-conjugated secondary antibody. After three additional washing steps, detection was performed using the SuperSignal West Femto chemiluminescence kit (Pierce).

FIG. 3A shows that a strong reduction in Ren-Luc activity occurs in receptor cells when RVFV-specific antisera were used. By contrast, a control antibody directed against the La Crosse virus envelope protein as well as sera from an uninfected mouse or individual had no neutralising activity.

The protein composition of VLPs was determined. To this aim, supernatants from 293T cells transfected with different plasmid combinations were purified and subjected to Western blot analysis (FIG. 3B). Supernatants from RVFV-infected cells served as positive control (lane 1). As expected, there was no release of viral proteins from minireplicon-expressing cells lacking glycoprotein expression (lane 3). In contrast, both the Gn and N protein could be detected in supernatants where the full set of plasmids for the VLP system had been transfected (lane 2), indicating that the composition of VLPs is similar to that of RVFV particles. We noted, however, that the relative proportion of the 78 kD protein (which is recognized by the monoclonal antibody against Gn) was substantially higher in VLPs when compared to virus particles. Apparently, expression or particle incorporation of the M segment-encoded proteins differs between plasmid-transfected and virus-infected cells. Together, these data indicate that the VLPs generated by coexpression of the minireplicon system and the M-encoded glycoproteins can serve not only as a model for authentic RVFV particles, but also for immunization of humans.

### EXAMPLE 4

### Concentration and Optimization of RVF Virus-Like Particles

### a) Concentration

Supernatants from VLP-expressing cells were collected and clarified from cell debris by centrifugation (12.000×g, 10 min at 4° C.). The particles were then concentrated by ultracentrifugation at 24,000 rpm and 4° C. for 2 h in a Beckman SW41Ti rotor. The pellet was dried for 5 min before resuspension in phosphate-buffered saline (PBS).

Concentrated VLPs were used in the Western Blot shown in FIG. 3B and for vaccination.

### b) Optimization of VLP Production

A time course analysis was performed to study the kinetics of VLP formation. Donor cells were transfected with either the minireplicon alone, or in addition with the M-expression construct to generate VLPs. From parallel dishes, cell lysates and supernatants were harvested after 24, 48 or 72 hours. Reporter assays of donor cell lysates showed again (compare FIG. 2 B, left panel) that addition of the M segment expression plasmid had no substantial effect on minireplicon activity (FIG. 4A, columns 2 and 3). Both the minireplicon and the VLP donor cells displayed peak activity at 48 h post-transcription, and a decrease at the 72 h time point.

This indicates robust and continued production of recombinant RVFV components and, most probably, exhaustion after longer expression times. When supernatants of the time course were used to infect indicator cells, reporter assays confirmed again that the M segment expression plasmid was necessary to generate infectious VLPs (FIG. 4B). Moreover, VLP activities steadily increased in the supernatants harvested from 24 h to 72 h after transfection of donor cells. The apparent discrepancy between donor cells (peak at 48 h time point) and indicator cells (steady increase until 72 h) may simply be due to the fact that VLPs are relatively stable and accumulate in the supernatants over the whole time course, whereas gene expression in donor cells fades out.

Therefore, defined conditions for the highly efficient production of VLPs are disclosed.

### EXAMPLE 5

### Primary Transcription of Genes Packaged in VLPs

Conventionally, primary transcription of negative-strand RNA viruses is measured by using translational inhibitors, e.g. cycloheximide. Cutting off the supply of newly synthesized viral proteins allows to measure the activity of incoming polymerase complexes without the amplification loop established by genome replication. However, the fact that bunyavirus mRNA transcription is strictly dependent on concurrent translation precludes a dissection of primary and secondary transcription by pharmaceutical means.

It was checked whether the VLP system would allow the measurement of RVFV primary transcription. Thus, BSR-T7/5 cells were infected with VLPs and their transcriptional activity was monitored by measuring Ren-Luc over a period of 48 h. To distinguish between primary and secondary transcription, the indicator cells were either left untransfected or were transfected with polymerase complex proteins N and L prior to infection (FIG. 5). In parallel, supernatants from minireplicon-expressing cells were used to detect potential background reporter activity. As expected, strong Ren-Luc activity was observed in VLP-infected cells where polymerase complexes were present (FIG. 5A). This activity steadily increased until 48 h post-infection, the last timepoint measured, thus being indicative for continuous replication and transcription. Supernatants from minireplicon-only transfected cells, by contrast, did not elicit any detectable Ren-Luc activity. Interestingly, a clear and reproducible Ren-Luc activity over background was also measured in VLP-infected naïve (untransfected) cells, albeit to a much lower extent than in N and L-expressing cells. Maximum activity was reached after 24 h and remained at an almost constant level for further 24 h. This indicates that the VLP-associated viral polymerase is highly active in primary transcription. The same observation was also made in several other cell lines infected with VLPs. The established system thus allows, for the first time, to study primary transcription of a bunyavirus.

### EXAMPLE 6

### Effect of the Antiviral Protein MxA on VLP Transcription

The VLP system of the present application offers the chance to dissect the effect of the antivirally active protein MxA on primary and secondary transcription of bunyaviruses. To investigate this, indicator cells were transfected with an expression construct for MxA and infected with RVFV VLPs. The MxA mutant T103A served as a negative control. FIG. 6A (upper part) shows that MxA expressed by indicator cells was capable of reducing the reporter activity of incoming VLPs, indicating an effect on primary transcription. Moreover, if N and L support constructs were expressed in addition to MxA, a similar, but somewhat stronger inhibitory effect was observed, although the VLP reporter activity was much higher as compared to the primary transcription set-up (FIG. 6B, upper part). Western blot analyses for MxA and RVFV N were performed to control recombinant gene expression (FIGS. 6A and B, lower parts). These data suggest that MxA is capable of interfering with primary transcription of bunyaviruses and hence interacts not only with the soluble N protein, but also with assembled RNPs.

In summary, reverse genetics tools have been established which enable the study of RVFV RNP activity, virus assembly, virus entrance, primary transcription and antiviral mechanisms under non-BSL-3 conditions. Moreover, the modular buildup of those systems allows to dissect the viral life cycle in a systematic manner.

### EXAMPLE 7

### Vaccination of Mice

VLPs according to the teaching of the present application whereby the VLPs contained the gene coding for N instead of the Renilla reporter gene (so-called N-VLPs) were produced and concentrated as described in Example 3 and 4a).

Two groups of six mice each (C57/BL6) were immunized intraperitoneally with either PBS (as control) or with 1,000,000 N-VLPs.

Two weeks after the immunization both groups of mice were challenged with 100,000 pfu (plaque-forming units) of the Rift Valley Fever Virus strain ZH548 intraperitoneally.

In the control group (PBS) five of six mice died.

From the group of mice immunized with N-VLPs according to the present invention all six mice survived the viral challenge.

It is very surprising that after a single vaccination a protection could be obtained after 14 days. Alternatively it is possible to repeat the immunization in such a manner that the animal is immunized two or three times after suitable periods of time ranging between two weeks and three months.

In the experiment a plasmid called pHH21_RVFV_vM-N was constructed containing the N-ORF of RVFV in a negative orientation flanked by the UTRs of the M segment. The N-ORF has been amplified by using appropriate primers.

### EXAMPLE 8

### Increasing VLP Yields

The inhibition of the antivirally active kinase PKR increases VLP yields to a significant extent. PKR is activated by viral double-stranded RNA (dsRNA) or single-stranded RNA (ssRNA) containing a 5′ triphosphate group and a short stem-loop structure (Sadler and Williams. Nat Rev Immunol 8:559-68 (2008)). PKR is a serine-threonine kinase that phosphorylates the alpha subunit of the eukaryotic translation initiation factor elF2, thereby leading to a translational arrest of cellular and viral mRNA translation. Some cells express a natural dominant-negative mutant of PKR called PKRDeltaE7 (Li, S. and A. E. Koromilas. J Biol Chem 276:13881-90 (2001). The expression of the VLP constructs as well as the activity of VLPs could be affected by PKR, since transfection and virus replication are known to activate PKR. Therefore, PKRDeltaE7 was used and this potent PKR inhibitor was coexpressed with the constructs for VLP production. The expression construct for PKRDeltaE7 was made by amplifying the cDNA Sequence by RT-PCR and cloning into the high-level expression vector pl.18 (which is also used for the VLP constructs). The experiment was performed as follows:

a) Subconfluent monolayers of 293T cells seeded in 6-well plates (donor cells) were transfected with 0.5 μg of plasmids pl.18_RVFV_N, pl.18_RVFV_L, pl.18_RVFV_M, pHH21_RVFV_vMRen, and with 0.1 μg of pGL3-control using Fugene HD transfection reagent. If required, 0.5 μg of pl.18_PKRDeltaE7 were added to the transfection mix. At 48 h post-transfection, supernatants were harvested and donor cells were lysed with 200 μl Passive Lysis Buffer to measure luciferase activities as described above. The supernatants were treated with 1 μl/ml Benzonase (Novagen) at 37° C. for 3 h and centrifuged at 12,000×g for 5 min to remove residual plasmids and cellular debris. Aliquots of the treated supernatants were then used to infect BSR-T7/5 cells (indicator cells). Replication and transcription of VLPs was supported by transfecting indicator cells with N and L expression plasmids pl.18_RVFV_N and pl.18_RVFV_L (0.5 μg each) 16 h prior to the transfer of supernatant.
b) The results in FIG. 7 show that co-transfection of 0.5 μg pl.18_PKRDeltaE7 in donor cells increases reporter activity in donor cells by a factor of about 5 (compare reporter activity of columns 2 and 3 in A vs. B). Importantly, co-transfection of pl.18_PKRDeltaE7 increased VLP production by a factor of about 10 (indicator cells, column 3, A vs. B). These results clearly indicate that PKR is inhibitory to VLP production, and that yields can be dramatically increased by blocking PKR.

Further experiments have shown that PKRDeltaE7 enhances the performance of reverse genetics systems of bunyaviruses in general.

### EXAMPLE 9

### Increase of the Autonomous VLP Transcription and Immunogenicity

The viral genomic S segment of RVFV codes for two different viral genes (N and NSs) by a so-called ambisense-strategy. The N is transcribed like the other RVFV genes in minus-strand orientation, i.e. in a conventional manner. The NSs gene, by contrast, is transcribed from the other direction (FIG. 8 A). We exploited this dual expression strategy to produce VLPs which express the N together with another gene of interest (replacing NSs). FIG. 8 B shows the expression strategy of the VLPs described up to here, which express a single gene of interest (Ren-Luc or N), compared with the ambisense (Ambi-) VLPs.

The co-expressed N Gen of the ambisense VLPs is useful for two different purposes. On one hand increases N (which is part of the viral polymerase complex) the autonomous VLP expression (see below), on the other hand increases N expression the immunogenicity of the VLPs. The N protein of the RVFV-related Toscana virus is known to act as CTL antigen and may even induce neutralizing antibodies (Savellini et al., Virology 375:521-8 (2008)).

A comparison of the Renilla-activity of conventional Ren-VLPs with Ambi-VLPs demonstrates the enhancing effect of N expression (FIG. 9). The Ambi-VLP construct (vS-N-Ren) has in donor cells a lower basic activity as the M segment-based Ren-VLPs (FIG. 9 A). The reason for this is that the S segment promoter used for making the Ambi-VLPs has a lower basic activity as compared to the M promoter used for generating the Ren-VLPs (Gauliard et al., Virology 351:170-9 (2006)). Nonetheless show the Ambi-VLPs an approximately 5-fold stronger activity in indicator cells as Ren-VLPs, and even 3 days after VLPs infection there is still detectable activity (FIG. 9 B). Thus, the co-expression of the N gene on the Ambi-VLPs leads to a higher and more persevering VLP activity. To our knowledge, such an ambisense-VLP-System was not described so far.

Potential applications of the Ambi-VLP-System are: Marker vaccine (e.g. Renilla as Marker to distinguish infected from vaccinated animals), overexpression of immunity-enhancing cytokines, expression of small interfering RNAs, Expression of other viral proteins.

1. A method for producing virus-like particles comprising the steps of:
a) transfecting a mammalian cell line with several independent vectors comprising
aa) a vector containing at least a substantial part of the M gene from Rift Valley Fever Virus,
bb) a vector containing at least a substantial part of the L gene from Rift Valley Fever Virus,
cc) a vector containing at least a substantial part of the N gene from Rift Valley Fever Virus and
dd) a vector containing a multicloning site flanked by the non-coding 5′- and 3′-ends of the L-, M- or S-segment of Rift Valley Fever Virus, and
b) culturing the transfected mammalian cell line under conditions suitable for generating virus-like particles (VLPs); and
c) obtaining the VLPs which are capable of autonomous gene expression from the supernatant of the transfected cultured cell lines.

2. The method according to claim 1 wherein the mammalian cell line is a human cell line.

3. The method according to claim 1 wherein the vector containing the non-coding 5′- and 3′-ends of the L-, M- or S-segment of the Rift Valley Fever Virus and a multicloning site contains an additional gene inserted within the multicloning site.

4. The method according to claim 3 wherein a gene coding for luciferase obtained from Renilla is inserted within the multicloning site.

5. The method according to claim 3 wherein a gene coding for the N protein of Rift Valley Fever Virus has been inserted within the multicloning site.

6. The method according to claim 3 wherein the gene coding for the L protein of Rift Valley Fever Virus has been inserted within the multicloning site.

7. The method according to claim 1 wherein a dominant-negative mutant of the antivirally active kinase PKR is added during step a).

8. The method according to claim 1 wherein the vector mentioned under dd) is replaced by a vector containing at least a substantial part of the N-gene and a multicloning site.

9. A virus-like particle comprising at least one protein of the Rift Valley Fever Virus, wherein said particle is obtained by the method according to claim 1.

10. A pharmaceutical composition comprising the virus-like particle according to claim 9.

11. The pharmaceutical composition of claim 10, wherein said composition comprises a vaccine against an infection of Rift Valley Fever Virus suitable for human administration.

12. The pharmaceutical composition of claim 10, wherein said composition comprises a vaccine against an infection of Rift Valley Fever Virus suitable for human administration.

13. A method of testing an agent for antiviral activity against Rift Valley Fever Virus, said method comprising the steps of:
a) bringing the virus-like particles according to claim 9 into contact with a mammalian cell line and an agent to be tested for antiviral activity and
b) measuring the antiviral activity of the test agent through comparison with suitable control which does not have antiviral activity.

14. The method according to claim 13 wherein the agent to be tested is selected from the group comprising anti-RVFV antibodies or fragments thereof, chemically synthesized compounds supposed to have antiviral activity or proteins, peptides, steroids of natural origin and derivatives thereof supposed to have antiviral activity against Rift Valley Fever Virus.

15. A test kit for testing an agent for antiviral activity against Rift Valley Fever Virus, characterized in that the kit comprises a mammalian cell line and virus-like particles according to claim 9 and the test steps comprise (a) bringing the mammalian cell line and virus-like particles according to claim 9 into contact with an agent to be tested for antiviral activity and (b) measuring the antiviral activity of the test agent through comparison with a suitable control that does not have antiviral activity.
